(19) United States
(12) Patent Application Publication
(10) Pub. No.: US 2016/0303256 A1
(43) Pub. Date: Oct. 20, 2016

(54) METHODS AND COMPOSITIONS FOR SELF-ASSEMBLY SYSTEM OF NANOPARTICLES AND MICROPARTICLES FOR MULTI-TARGETING SPECIFICITY

(71) Applicant: THE UNIVERSITY OF NORTH CAROLINA AT CHAPEL HILL, Chapel Hill, NC (US)

(72) Inventor: Rihe Liu, Chapel Hill, NC (US)

(21) Appl. No.: 15/026,165

(22) PCT Filed: Sep. 30, 2014
(86) PCT No.: PCT/US2014/058257

§ 371 Date: Mar. 30, 2016

Related U.S. Application Data
(60) Provisional application No. 61/884,609, filed on Sep. 30, 2013.

(51) Int. Cl. A61K 49/00; C07K 17/10; G01N 33/543; A61K 47/48

(52) U.S. Cl. CPC A61K 49/0004; A61K 47/48853; C07K 17/10; G01N 33/54346; G01N 2333/4727; G01N 2800/7028

(57) ABSTRACT

The present invention provides a nanoparticle or microparticle comprising calmodulin attached to an exterior surface, wherein the calmodulin is attached to a fusion protein comprising a targeting ligand and a carboxy-terminal or amino-terminal calmodulin binding peptide and methods of its use in diagnostics and therapeutics.

### STATEMENT OF PRIORITY

This application is a 35 U.S.C. §371 national stage application of International Application No. PCT/US2014/058257, filed Sep. 30, 2014, which claims the benefit, under 35 U.S.C. §119(e), of U.S. Provisional Application Ser. No. 61/884,609, filed Sep. 30, 2013, the entire contents of each of which are incorporated by reference herein.

### STATEMENT OF GOVERNMENT SUPPORT

This invention was made with government support under Grant Nos. CA157738 and CA151652 awarded by the National Institutes of Health. The government has certain rights in the invention.

### STATEMENT REGARDING THE ELECTRONIC FILING OF A SEQUENCE LISTING

A sequence listing in ASCII text format, submitted under 37 C.F.R. §1.821, entitled 5470-670 ST25.txt, 12,157 bytes in size, generated on Jul. 8, 2016, and filed electronically via EFS-Web, is provided in lieu of a paper copy.

### FIELD OF THE INVENTION

The present invention relates to a universal self-assembly system to confer particles (e.g., nanoparticles or microparticles) with desired multi-targeting specificity for diagnosis and therapeutics.

### BACKGROUND OF THE INVENTION

Cancers are highly polygenetic and different malignant tumors that have very different biomarker expression profiles. In the case of small cell lung cancers, for example, more than 23,000 mutations are exclusive to the diseased cells. Such a polygenetic nature of most cancers dictates that any effective cancer therapy should be based on targeting multiple pertinent signaling pathways, presumably by using a combination of targeted therapeutics. Considering all imaging tracers and drug carriers as a cargo, tumor cells expressing a specific biomarker can be targeted with a cargo functionalized with respective targeting ligands (TLs). Restricting a cytotoxic drug to the diseased site(s) in this manner has the benefit of reducing serious side effects on healthy tissues, increasing local drug concentrations, and improving efficacy of treatment. One major barrier in the development of nano- or micro-carriers for imaging or therapeutic agents is the oriented installment of targeting molecules that allow for targeted delivery of bioconjugates to diseased tissues. Regardless of the materials used to compose the particles, one vital challenge is how to conjugate targeting ligands (TLs) to the surface while precisely controlling the proportion of different ligands if multispecificity is desired. Ideally, the multispecificity and targeting properties of one kind of prepared particles would be easily tunable by simply changing the types and proportions of TLs bound to it. Such particles loaded with multiple TLs could have greatly expanded applications to different tumor types and better therapeutic and diagnostic efficacies. However, conjugating particles with a single TL while still maintaining its target-binding ability is already challenging, let alone using multiple TLs, each possessing a unique specificity. The goal of introducing multispecific targeting features to nano- or micro-particles identifies an unmet need.

The present invention overcomes previous shortcomings in the art and addresses this unmet need by providing methods and compositions for a universal self-assembly system to confer nano- or micro-particles with desired multi-targeting specificity for diagnosis and therapeutics.

### SUMMARY OF THE INVENTION

The present invention provides a particle (e.g., a nanoparticle or microparticle) comprising calmodulin attached to an exterior surface, wherein the calmodulin is attached to a fusion protein comprising a targeting ligand and a carboxy-terminal or amino-terminal calmodulin binding peptide.

Also provided herein is a method of making the particle of this invention, comprising the calmodulin/fusion protein complex by a self-assembly process in the presence of calcium.

In addition, the present invention provides a method of delivering a therapeutic agent to a cell of a subject, comprising administering to the subject the particle of this invention, wherein the particle comprises a therapeutic agent and where the particle further comprises a targeting ligand specific for the cell of the subject to which the therapeutic agent is to be delivered.

Furthermore, the present invention provides a method of detecting the presence and/or location of a target cell (e.g., a cancer cell) in a subject, comprising administering to the subject the particle of any preceding claim wherein the particle comprises a detectable agent and where the particle further comprises a targeting ligand specific for the target cell.

Additionally provided herein is a method of treating cancer in a subject in need thereof, comprising administering to the subject the particle of any preceding claim, wherein the particle comprises a chemotherapeutic agent and/or anti-cancer agent and where the particle further comprises a targeting ligand specific for the cancer cell of the subject to which the chemotherapeutic agent and/or anti-cancer agent is to be delivered, thereby treating cancer in the subject.

The present invention also provides a method of diagnosing cancer in a subject, comprising administering to the subject the particle of any preceding claim, wherein the targeting ligand on the particle is specific for a target molecule on a cancer cell in the subject and the particle further comprises an imaging molecule and/or detectable molecule, whereby the targeting ligand binds the target molecule on a cancer cell in the subject and the imaging molecule is visualized and/or the detectable molecule is detected on a cancer cell in the subject, thereby diagnosing cancer in the subject.

### BRIEF DESCRIPTION OF THE DRAWINGS

FIG. 1. Schematic of the self-assembly of targeting ligands with different specificities on the surface of nanocarriers. Nanocarriers are coated with uapuapuapu using Bis(Sulfosuccinimidyl) suberate (BS3). Recombinant single domain targeting ligands with the C-terminal universal adaptor peptide self-assemble on the nanocarrier by binding to calmodulin in the presence of Ca2+. This self-assembly is accompanied by a conformation change in calmodulin and is reversible by reducing the concentration of Ca2+.

FIG. 2. Graphical representation of in vitro capture assay using calmodulin-coated agarose beads treated with FITC-labeled TL-UAPs. The fluorescence intensity was normalized with total input protein (IN) as 100%. A number of sample runs were conducted with different bead preparation protocols for each of the three TL-UAPs: a standard incubation of the beads with TL-UAPs in the presence of CaCl2 (S), standard incubation in the presence of a 100-fold excess of the MLCK-derived UAP as competitor (S+UAP), standard incubation in the absence of CaCl2 (S—CaCl2), standard incubation followed by washing with the washing buffer one, two and three times (W1-W3) and elution with washing buffer plus EGTA five times (E1-E5).

FIG. 3. Staining of fixed cancer cells using different TLs assembled on QD605-CaM. Nuclei were stained by DAPI. EGFR-positive A431 and HER2-positive HTB77 cells were selectively stained by corresponding TLs. Pictures were merged from two channels.

FIG. 4. Flow cytometry live cell-binding analysis using TLs assembled on QD605-CaM as compared with various control experiments. Binding of particular assembled QD605-CaM on a given biomarker-positive cell line led to a peak shift in flow cytometry.

FIGS. 5A-C. A) Flow cytometry cell-binding analysis using engineered K562 cells (EGFR−, HER2+, αvβ3+) treated with QD605-CaM onto which two different TLs have been assembled. Different combinations are shown, and Median Fluorescence Intensity (MFI) is calculated by peak position. B) Image of Z domain and FN3 domain are adopted from PDB 2KZJ and 1FNA. Active sites are labeled by black circles, introduced UAPs are presented by black dotted line. C) Flow cytometry cell-binding analysis using HTB77 cells treated with QD605-CaM@ZEGFR-UAP, ZEGFR-UAP pre-saturated CaM-QD605 followed by additional 1 h incubation with ZHER2-UAP or FN3αvβ3-UAP.

FIG. 6. SDS-PAGE electrophoresis analysis of purified targeting proteins. Molecular weights of the protein markers were labeled on the left side in kilodaltons. The gels were stained by Commassie Blue.

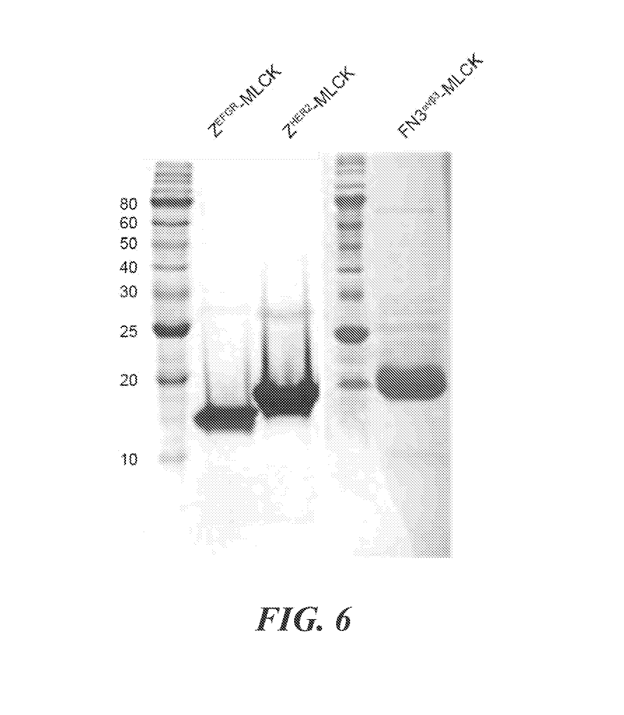

FIG. 7. Agarose electrophoresis analysis of 1) unmodified QD605; 2) QD605 conjugated with calmodulin. Gel was running in TAE buffer at 100 V until reached well separation and visualized under UV light.

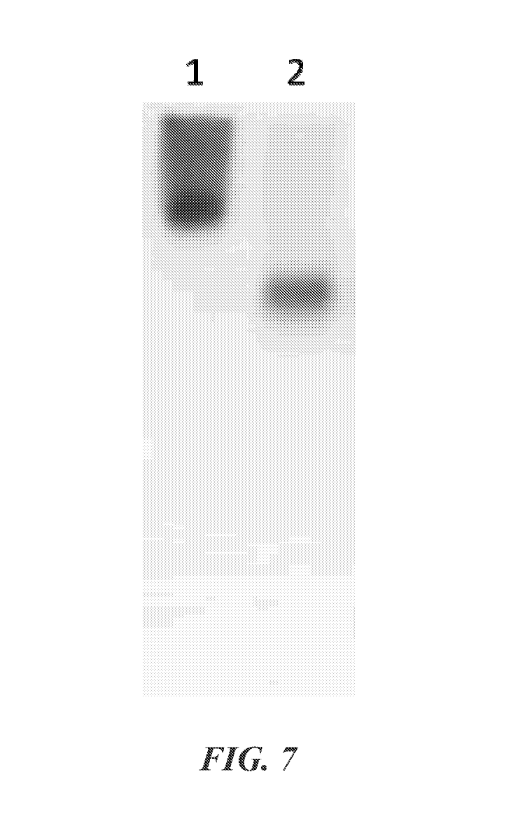

FIG. 8. Conjugation of calmodulin to nanoparticles and self-assembly of the targeting ligand(s) on nanoparticles.

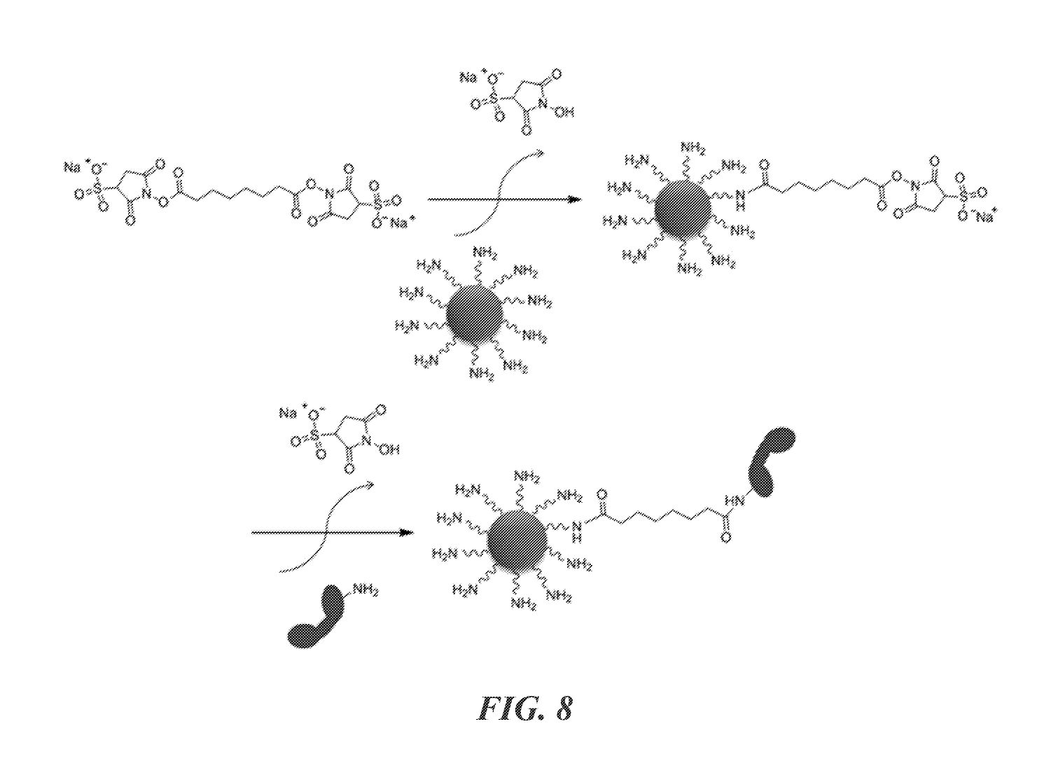

### DETAILED DESCRIPTION OF THE INVENTION

The present invention will now be described with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

As used herein, “a,” “an” and “the” can mean one or more than one, depending on the context in which it is used. For example, “a” cell can mean one cell or multiple cells.

Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a compound or agent of this invention, dose, time, temperature, and the like, is meant to encompass variations of ±20%, ±10%, ±5%, ±1%, ±0.5%, or even ±0.1% of the specified amount.

As used herein, the transitional phrase “consisting essentially of” means that the scope of a claim is to be interpreted to encompass the specified materials or steps recited in the claim, “and those that do not materially affect the basic and novel characteristic(s)” of the claimed invention. See, In re Herz, 537 F.2d 549, 551-52, 190 USPQ 461, 463 (CCPA 1976) (emphasis in the original); see also MPEP §2111.03. Thus, the term “consisting essentially of” when used in a claim of this invention is not intended to be interpreted to be equivalent to “comprising.”

The present invention is described in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure that do not depart from the instant invention. Hence, the following specification is intended to illustrate some particular embodiments of the invention, and not to exhaustively specify all permutations, combinations and variations thereof.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed.

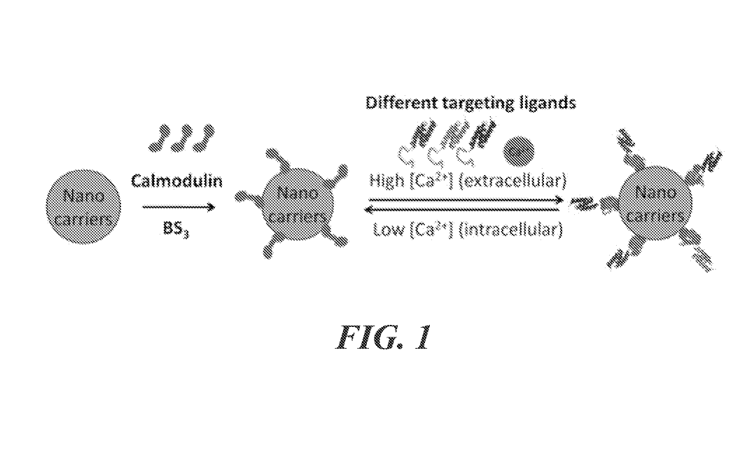

The present invention is directed to the unexpected discovery of an efficient and universal self-assembly system that confers particles (e.g., nanoparticles and/or microparticles) with desired multispecificity for tumor diagnosis and therapeutics. This system, summarized in FIG. 1, involves a universal acceptor (UA, i.e., calmodulin) and a universal adaptor peptide (UAP, e.g., a calmodulin binding protein such as a peptide fragment of human myosin light-chain kinase).

Thus, in one embodiment, the present invention provides a particle, which can be a nanoparticle or a microparticle, comprising calmodulin attached to an exterior surface of the particle, wherein the calmodulin is also noncovalently attached to a fusion protein comprising a targeting ligand and a carboxy-terminal or amino-terminal calmodulin binding peptide.

Types of nanoparticles of this invention include but are not limited to, polymer nanoparticles such as PLGA-based, PLA-based, polysaccharide-based (dextran, cyclodextrin, chitosan, heparin), dendrimer, hydrogel; lipid-based nanoparticless such as lipid nanoparticles, lipid hybrid nanoparticles, liposomes, micelles; inorganics-based nanoparticles such as superparamagnetic iron oxide nanoparticles, metal nanoparticles, platin nanoparticles, calcium phosphate nanoparticles, quantum dots; carbon-based nanoparticles such as fullerenes, carbon nanotubes; and protein-based complexes with nanoscales.

Types of microparticles of this invention include but are not limited to particles with sizes at micrometer scale that are polymer microparticles including but not limited to, PLGA-based, PLA-based, polysaccharide-based (dextran, cyclodextrin, chitosan, heparin), dendrimer, hydrogel; lipid-based microparticless such as lipid microparticles, micelles; inorganics-based microparticles such as superparamagnetic iron oxide microparticles, platin microparticles and the like as are known in the art.

As used herein, the terms “nanoparticle” and “nanosphere” describe a polymeric particle or sphere in the nanometer size range. The term microparticle” or “microsphere” as used herein describes a particle or sphere in the micrometer size range. Both types of particles or spheres can be used as drug carriers into which drugs, imaging agents and/or antigens may be incorporated in the form of solid solutions or solid dispersions or onto which these materials may be absorbed, encapsulated, or chemically bound.

A nanoparticle or nanosphere of this invention can have a diameter of 100 nm or less (e.g., in a range from about 1 nm to about 100 nm). In some embodiments, a particle with dimensions more than 100 nm can still be called a nanoparticle. Thus, an upper range for nanoparticles can be about 500 nm. A microparticle or microsphere of this invention can have a diameter of about 0.5 micrometers to about 100 micrometers.

In some embodiments, in the particle (e.g., nanoparticle or microparticle) of this invention, the calmodulin is attached to the exterior surface using hydrophobic noncovalent interaction or covalent linkage based on amine/carboxylate chemistry, thiol/maleimide chemistry, and disulfide chemistry. For hydrophobic noncovalent interaction, unmodified wild-type calmodulin is directly absorbed on the surface of particles. Alternatively, calmodulin is first chemically or enzymatically modified by conjugation with a fatty acid (i.e., lauric acid, myristic acid, palmitic acid, stearic acid, arachidic acid, oleic acid, etc.), whose long carbon chain allows for tight and strong hydrophobic interaction with or insertion into the surface of particles. For covalent linkage, the functional groups on the surface of particles are first derivatized or activated to introduce activated ester, activated disulfide, or maleimide, followed by reaction with wild-type calmodulin or genetically engineered thiol-containing recombinant calmodulin, respectively. The amino acid sequence of human calmodulin is provided under GenBank® Database Accession No. AAD45181.1, incorporated by reference herein.

In some embodiments, the calmodulin tightly binds to a calmodulin-binding peptide fused (e.g., at the amino acid sequence level) at the amino- or carboxy-terminal of a targeting ligand in the presence of calcium (Ca²⁺). The range of Ca²⁺ concentration can be from about 1 nM to about 10 mM (e.g., 0.5 nM, 1.0 nM, 1.5 nM, 20 nM, 2.5 nM, 3.0 nM, 3.5 nM, 4.0 nM, 5.0 nM, 5.5 nM, 6.0 nM, 6.5 nM, 7.0 nM, 7.5 nM, 8.0 nM, 8.5 nM, 9.0 nM. 9.5 nM, 10 nM, 100 nM, 200 nM 300 nM, 500 nM 1 mM, 2 mM, 3 mM, 4 mM, 5 mM, 6 mM, 7 mM, 8 mM, 9 mM, 10 mM, as well as any value in between these numbers not explicitly stated here).

In some embodiments, a flexible linker can be engineered between the targeting ligand and the calmodulin binding peptide, to facilitate interaction between the targeting ligand and its cell surface target. Nonlimiting examples of a linker of this invention include: a) a linker comprising, consisting essentially of or consisting of the amino acid sequence of SEQ ID NO:1 (GPQPQPKPQPK); b) a linker comprising, consisting essentially of or consisting of the amino acid sequence of SEQ ID NO:2 ((GGGGS)ₙ, wherein n can be any number such as 1, 2, 3, 4, 5, 6, 7, 8, 9, 10, etc.; e.g., (GGGGS)₃); c) a linker comprising, consisting essentially of or consisting of the amino acid sequence of SEQ ID NO:3 (TPPTPSPSTPPTPSP; human IgA1 heavy chain); d) a linker comprising, consisting essentially of or consisting of the amino acid sequence of SEQ ID NO:4 (EFPKPSTPPGSSGGAP; murine IgG3-hinge region); e) a linker comprising, consisting essentially of or consisting of the amino acid sequence of SEQ ID NO:5 (PQPQPQPKPQPKPEPE; camel IgG); f) a linker comprising, consisting essentially of or consisting of the amino acid sequence of SEQ ID NO:6 ((GGGS)ₙ, wherein n can be 1, 2, 3, 4, 5, 6, 7, 8, 9, 10, etc.); g) a linker comprising, consisting essentially of or consisting of the amino acid sequence of SEQ ID NO:7 ((GSGSGS)ₙ, wherein n can be 1, 2, 3, 4, 5, 6, 7, 8, 9, 10, etc.); h) a linker comprising, consisting essentially of or consisting of the amino acid sequence of SEQ ID NO:8 ((TPPTPSP)ₙ, wherein n can be any number such as 1, 2, 3, 4, 5, 6, 7, 8, 9, 10, etc.); i) a linker comprising, consisting essentially of or consisting of the amino acid sequence of SEQ ID NO:9 ((PQPQPK)ₙ, wherein n can be any number such as 1, 2, 3, 4, 5, 6, 7, 8, 9, 10, etc.); j) a linker comprising, consisting essentially of or consisting of the amino acid sequence of SEQ ID NO:10 ((PQPQPE)ₙ, wherein n can be any number such as 1, 2, 3, 4, 5, 6, 7, 8, 9, 10, etc.); k) a linker comprising, consisting essentially of or consisting of the amino acid sequence of SEQ ID NO:11 (PEPEPQPQGG); and 1) any combination of (a)-(k) above.

The linker peptide of this invention can also be a peptide of about 5 to about 50 amino acids (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 56, 47, 48, 49, or 50) having an amino acid composition that provides for the functional feature of having the appropriate length and flexibility to facilitate the positioning of the target binding domains for binding at their respective sites on the target molecule(s) on the cell surface.

UAP stands for “universal acceptor peptide” such as a calmodulin-binding peptide that can be, for example, present in nature or isolated from a combinatory library (Biochemistry, 2007, 46 (35), pp 10102-10112). Nonlimiting examples of UAP from organisms include RRKWQKTGHAVRAIGRLSSM (SEQ ID NO:12) from MLCK; NSAFVERVRKRGFEVV (SEQ ID NO:13) from HSP90; WSRIASLLHRKSAKQCKAR (SEQ ID NO:14) from CDC5-L; YEAHKRLGNRWAEIAKLLP (SEQ ID NO:15) from MYBL1; KEVIRNKIRAIGKMARVFSV (SEQ ID NO:16) from PPP3C; and ELRSLWRKAIHQQILLLR (SEQ ID NO:17) from TBC1. Nonlimiting examples of UAP from combinatorial libraries include KSIIQRNLRWNKFKRFYQD (SEQ ID NO:18); NILRQEVMKMGPAKDTVRN (SEQ ID NO:19); WVKLRQRVTLAKRVAVNLNY (SEQ ID NO:20); LRLVPRIKALNKVQVKNHN (SEQ ID NO:21); WINNVRLRIHTKRWLLKSNH (SEQ ID NO:22); and WHKVFIRRQSKKLVYNTIKN (SEQ ID NO:23).

Further nonlimiting examples of a calmodulin-binding peptide of this invention include a peptide comprising, consisting essentially of or consisting of the amino acid sequence RRKWQKTGHAVRAIGRLSSM (SEQ ID NO:12) from smooth muscle myosin light-chain kinase (MLCK); a peptide comprising, consisting essentially of or consisting of the amino acid sequence KRRWKKNFIAVSAANRFKKI (SEQ ID NO:24) from skeletal muscle MLCK; a peptide comprising, consisting essentially of or consisting of the amino acid sequence RRKLKGAILTTMLATR (SEQ ID NO:25) from Ca²⁺/CaM-dependent protein kinase; a peptide comprising, consisting essentially of or consisting of the amino acid sequence IPSWTTVILVKSMLRKRSFGNPF (SEQ ID NO:26) from Ca²⁺/CaM-dependent protein kinase; a peptide comprising, consisting essentially of or consisting of the amino acid sequence QILWFRGLNRIQTQIRVVNA (SEQ ID NO:27) from plasma membrane calcium-ATPase; a peptide comprising, consisting essentially of or consisting of the amino acid sequence ITRIQAQSRGVLARMEYKKL (SEQ ID NO:28) from beta myosin heavy chain; a peptide comprising, consisting essentially of or consisting of the amino acid sequence ATLIQKIYRGWRCRTHYQLM (SEQ ID NO:29) from myosin IA; a peptide comprising, consisting essentially of or consisting of the amino acid sequence AAKIQASFRGHMARKKIKSG (SEQ ID NO:30) from neurogranin; a peptide comprising, consisting essentially of or consisting of the amino acid sequence AIIIQRAYRRYLLKQKVKKV (SEQ ID NO:31) from voltage-gated sodium channel; a peptide comprising, consisting essentially of or consisting of the amino acid sequence LGLVQSLNRQRQKQLLNENN (SEQ ID NO:32) or RLLWQT AVRHITEQRFIHGHR (SEQ ID NO:33) from adenylyl cyclase; a peptide comprising, consisting essentially of or consisting of the amino acid sequence NEELRAIIKKIWKRTSMKLL (SEQ ID NO:34) from L-type Ca2+ channel; a peptide comprising the amino acid sequence MRSVLISLKQAPLVH (SEQ ID NO:35) from clathrin light chain A; a peptide c comprising, consisting essentially of or consisting of the amino acid sequence ARKEVIRNKIRAIGKMARVFSVLR (SEQ ID NO:36) from calcineurin A; a peptide comprising, consisting essentially of or consisting of the amino acid sequence KPKFRSIVHAVQAGIFVERMFRR (SEQ ID NO:37) from phosphodiesterase 1B; a peptide comprising, consisting essentially of or consisting of the amino acid sequence SYEFKSTVDKLIKKTNLALV (SEQ ID NO:38) from sodium/calcium exchanger (SLC8A); a peptide comprising, consisting essentially of or consisting of the amino acid sequence HTLIKKDLNMVVSAARISCG (SEQ ID NO:39) from titin; a peptide comprising, consisting essentially of or consisting of the amino acid sequence EIRFTVLVKAVFFASVLMRK (SEQ ID NO:40) from inducible nitric oxide synthase; a peptide comprising, consisting essentially of or consisting of the amino acid sequence AIGFKKLAEAVKFSAKLMGQ (SEQ ID NO:41) from neuronal nitric oxide synthase; a peptide comprising, consisting essentially of or consisting of the amino acid sequence ASP WKSARLMVHTVATFNSIK (SEQ ID NO:42) from spectrin; a peptide comprising, consisting essentially of or consisting of the amino acid sequence NSAFVERVRKRGFEVV (SEQ ID NO:13) from heat shock protein 90 (HSP90); a peptide comprising, consisting essentially of or consisting of the amino acid sequence WSRIASLLHRKSAKQCKAR (SEQ ID NO:14) from cell cycle serine/threonine-protein kinase 5-like (CDC5-L); a peptide comprising, consisting essentially of or consisting of the amino acid sequence YEAHKRLGNRWAEIAKLLP (SEQ ID NO:15) from V-Myb Myeloblastosis Viral Oncogene Homolog like-1 (MYBL1); a peptide comprising, consisting essentially of or consisting of the amino acid sequence QKEVLITWDKKLLNCRAKIR (SEQ ID NO:43) from TBC1; a peptide comprising, consisting essentially of or consisting of the amino acid sequence KSIIQRNLRWNKFKRFYQD (SEQ ID NO:18); a peptide comprising, consisting essentially of or consisting of the amino acid sequence NILRQEVMKMGPAKDTVRN (SEQ ID NO:19); a peptide comprising, consisting essentially of or consisting of the amino acid sequence VKLRQRVTLAKRVAVNLNY (SEQ ID NO:44); a peptide comprising, consisting essentially of or consisting of the amino acid sequence LRLVPRIKALNKVQVKNHN (SEQ ID NO:21); a peptide comprising, consisting essentially of or consisting of the amino acid sequence WINNVRLRIHTKRWLLKSNH (SEQ ID NO:22); a peptide comprising, consisting essentially of or consisting of the amino acid sequence WHKVFIRRQSKKLVYNTIKN (SEQ ID NO:23), singly or in any combination thereof.

In some embodiments of this invention, the targeting ligand can be, but is not limited to, a single chain polypeptide of a V_{H} or V_{L} domain of an antibody, a peptide or protein derived from a binding and/or framework region of an antibody, a single domain antibody mimic based on a non-immunoglobulin scaffold (such as an FN domain-based monobody, Z domain-based affibody, DARPIN), a short target-binding peptide containing natural and/or unnatural amino acids, which specifically binds to the extracellular domain of a cell surface receptor, singly and in any combination.

Nonlimiting examples of a targeting ligand of this invention include FN3^{VEGFR}, FN3^{αvβ3}, FN3^{EGFR}, FN3^{HER2}, FN3^{HER3}, FN3^{PSMA}, FN3^{GRP78}, FN3^{c-MET}, FN3^{IGFIR}, Z^{EGFR}, Z^{HER2}, Z^{HER3}, singly or in any combination.

A particle of this invention can comprise a polymer that can be PLGA-based, PLA-based, and/or polysaccharide-based (dextran, cyclodextrin, chitosan, heparin etc.); a dendrimer; a hydrogel; a lipid base; a lipid hybrid base; a liposome; a micelle; an inorganic base such as, e.g., superparamagnetic iron oxide, metal, platin, calcium phosphate; a quantum dot; a carbon base, such as, e.g., a fullerene, a carbon nanotube; and a protein-based complex with nanoscales.

In some embodiments of this invention, the particle can comprise a therapeutic agent. Nonlimiting examples of a therapeutic agent of this invention include small molecule drugs such as camptothecin, gemcitabine, auristatin, maytansinoid, calicheamicin, taxoid, epothilone, vinblastine, cisplatin or their derivatives as are known in the art, nucleic acid drugs (siRNAs, shRNAs, miRNAs), peptide drugs, and protein drugs.

In some embodiments of this invention, the particle can comprise a detectable agent. Nonlimiting examples of a detectable agent of this invention include a radioisotope, an MRI contrast agent, a fluorescent near-IR fluorescent molecule or any combination thereof.

In some embodiments, the particle of this invention can comprise two or more (e.g., 2, 3, 4, 5, 6, 7, 8, 9, 10, etc.) different fusion proteins wherein each different fusion protein comprises a different targeting ligand and a carboxy-terminal or amino terminal calmodulin binding peptide and the different fusion proteins can be present in any combination or ratio. The carboxy-terminal or amino terminal calmodulin binding peptide fused to different targeting ligands is usually the same, which solely dictates the binding of the fusion proteins with calmodulin immobilized on the particles. Therefore, the multispecificity of the particles can be tuned by changing the proportions and ratios of the loading targeting ligands.

The present invention further provides a method of producing a particle comprising calmodulin attached to an exterior surface, wherein the calmodulin is noncovalently attached to a fusion protein comprising a targeting ligand and a carboxy-terminal or amino-terminal calmodulin binding peptide in a Ca²⁺ dependent manner, comprising: a) coating a particle (e.g., a nanoparticle or microparticle or any combination thereof) of this invention with calmodulin; b) contacting the coated particle of (a) with a fusion protein comprising a targeting ligand and carboxy-terminal or amino-terminal calmodulin binding peptide in the presence of calcium under conditions whereby the fusion protein of (b) binds the calmodulin on the particle of (a) by self assembly in the presence of calcium, thereby producing the particle comprising calmodulin attached to an exterior surface, wherein the calmodulin is attached to a fusion protein comprising a targeting ligand and a carboxy-terminal or amino-terminal calmodulin binding peptide.

Also provided herein is a method of making the particle of this invention, comprising the calmodulin/fusion protein complex by a self-assembly process in the presence of calcium. The calmodulin-containing particles are typically prepared by introducing calmodulin onto the surface of micro- or nano-particles by (but not limited to) the following approaches: 1) non-covalent coating of the particle surface through nonspecific interactions between particles and calmodulin protein; or 2) covalent chemical conjugation between functional groups on particles and calmodulin, respectively; or 3) self-insertion into the hydrophobic layer of particles (if they contain such a layer) through the lipid tail engineered at the terminus of calmodulin. The final particles-calmodulin/targeting protein complex can be prepared by mixing the calmodulin-containing particles with one or more than one targeting protein(s) in a physiological related buffer in the presence of Ca2+ ion at an appropriate concentration. The molar ratio of calmodulin-containing particles and total targeting ligand(s) is in the range from about 1:1 to about 1:10,000. The ratio among different targeting ligands is tuned to match the ratio of the corresponding biomarkers on the surface of cells. The concentration of Ca2+ is from about 25 nM to about 25 mM (e.g., about 25 nM, 50 nM, 75 nM, 100 nM, 200 nM, 250 nM 300 nM, 350 nM, 400 nM, 500 nM, 750 nM, 1.0 mM, 1.5 mM, 2.0 mM, 2.5 mM, 3.0 mM, 3.5 mM, 4.0 mM, 4.5 mM, 5.0 mM, 6.0 mM, 7.0 mM, 8.0 mM, 9.0 mM, 10 mM, 15 mM, 20 mM, 25 mM, etc., including any value within this range not specifically recited herein, and in some embodiments, is at physiological concentrations (e.g., about 2-2.5 mM).

The present invention also provides an isolated nucleic acid molecule encoding the targeting ligand and/or the fusion protein comprising a targeting ligand and a calmodulin binding protein of this invention, a vector comprising the nucleic acid molecule of this invention, a cell (e.g., an isolated cell and/or transformed cell) comprising the nucleic acid molecule of this invention and a cell (e.g., an isolated cell and/or transformed cell) comprising the vector of this invention.

In further embodiments, the present invention provides various methods employing the particles of this invention. Thus, in one embodiment, the present invention provides a method of delivering a therapeutic agent to a cell of a subject, comprising administering to the subject a particle of this invention, wherein the particle comprises a therapeutic agent and wherein the particle further comprises a targeting ligand specific for the cell of the subject to which the therapeutic agent is to be delivered. In some embodiments, the cell is a cancer cell and the therapeutic agent is a chemotherapeutic agent or other anti-cancer agents.

The present invention further provides a method of treating cancer in a subject (e.g., a subject in need thereof), comprising administering to the subject a particle of this invention, wherein the particle comprises a chemotherapeutic agent and/or other anti-cancer agent and wherein the particle further comprises one or more than one targeting ligand specific for a cancer cell of the subject to which the chemotherapeutic agent and/or other anti-cancer agent is to be delivered.

“Effective amount” or “treatment effective amount” as used herein refers to an amount of a protein, fragment, nucleic acid molecule, vector and/or composition of this invention that is sufficient to produce a desired effect, which can be a therapeutic effect and/or an improvement. Alternatively stated, a “treatment effective” or “effective” amount is an amount that will provide some alleviation, mitigation, decrease or stabilization in at least one clinical symptom/sign in the subject. Those skilled in the art will appreciate that the therapeutic effects need not be complete or curative, as long as some benefit is provided to the subject. The effective amount or treatment effective amount will vary with the age, general condition of the subject, the severity of the condition being treated, the particular compound, agent, substance or composition administered, the duration of the treatment, the nature of any concurrent treatment, the pharmaceutically acceptable carrier used if any, and like factors within the knowledge and expertise of those skilled in the art. As appropriate, an “effective amount” or “treatment effective amount” in any individual case can be determined by one of ordinary skill in the art by reference to the pertinent texts and literature and/or by using routine experimentation. (Remington, The Science And Practice of Pharmacy (20th ed. 2000)).

In some embodiments of the methods of this invention, the particle of this invention can further comprise a cytotoxic moiety that kills the cancer cell subsequent to binding of the targeting ligand to cancer cells in the subject. In various embodiments, the cytotoxic moiety can be but is not limited to a small molecule, isotope, drug-containing nanoparticle, protein toxin, nucleic acid-based therapeutic agents (e.g., siRNA, miRNA, antisense, anti-gene oligonucleotide, etc.), or any combination thereof. Nonlimiting examples of cytotoxic small molecules include auristatin E, gemcitabine, maytansinoids, SN-38, calicheamicin, taxoids, epothilones, vinblastine, breflate, depsipeptide, and jasplakinolide or their derivatives as are known in the art. Nonlimiting examples of radioisotopes include copper-67, yttrium-90, and indium-111. Nonlimiting examples of cytotoxic protein toxins include ricin, diphtheria toxin, colicin Ia, exotoxin A, abrin, and gelonin. Nonlimiting examples of nanoparticles include gold nanoparticles, magnetite nanoparticles, PLGA-based nanoparticles, and liposome nanoparticles. Nonlimiting examples of nucleic acid-based agents include siRNA, miRNA, antisense, anti-gene oligonucleotides, etc., as are well known in the art.

In additional embodiments, the present invention provides a method of detecting the presence and/or location of a target cell in a subject, comprising administering to the subject a particle of this invention wherein the particle comprises an imaging molecule and/or detectable agent and wherein the particle further comprises a targeting ligand specific for the target cell, whereby the targeting ligand binds the target cell in the subject and the imaging molecule is visualized and/or the detectable molecule is detected at its binding location on the target cell in the subject, thereby detecting and/or localizing the target cell in the subject. In some embodiments, the target cell can be a cancer cell and/or other pathologic cell.

The present invention further provides a method of detecting and/or localizing cancer cells in a subject, comprising administering to the subject a particle of this invention, wherein the particle comprises an imaging molecule and/or detectable molecule and wherein the particle further comprises one or more than one targeting ligand specific for a target molecule on a cancer cell in the subject, whereby the targeting ligand binds the target molecule on a cancer cell in the subject and the imaging molecule is visualized and/or the detectable molecule is detected at its binding location on a cancer cell in the subject, thereby detecting and/or localizing a cancer cell in the subject. As noted herein, in such a method of detecting and/or localizing cancer cells in the subject, the particle of this invention can also be simultaneously acting as a therapeutic agent to treat the cancer in the subject.

In further embodiments, the present invention provides a method of diagnosing cancer in a subject, comprising administering to the subject a particle of this invention wherein the targeting ligand(s) on the particle is or are specific for a target molecule on a cancer cell in the subject and the particle further comprises an imaging molecule and/or detectable molecule, whereby the targeting ligand binds the target molecule on a cancer cell in the subject and the imaging molecule is visualized and/or the detectable molecule is detected on a cancer cell in the subject, thereby diagnosing cancer in the subject.

In the methods described herein, the imaging molecule can be but is not limited to an MRI contrast agent, a radioisotope for PET and nuclear medicine (e.g., ⁶⁴Cu-ATSM, ¹⁸F-FDG, fluoride, FLT, FMISO, gallium, technetium-99m, etc.), a near-IR fluorescence molecule, a nanoparticle-containing imaging agent or any combination thereof.

Nonlimiting examples of a cancer of this invention include breast cancer, lung cancer, prostate cancer, colorectal cancer, bladder cancer, skin cancer, renal cancer, pancreatic cancer, lymphoma, leukemia, head and neck cancer, stomach cancer, ovarian cancer, uterine cancer, cervical cancer, brain cancer, esophageal cancer, stomach cancer, colon cancer, anal cancer, liver cancer, bone cancer and any combination thereof.

In some embodiments of the methods of this invention, the target molecule can be an extracellular domain of a cell surface receptor (such as epidermal growth factor receptor family members (EGFR, HER2, FlER3, HER4, etc.), c-MET, VEGFR, insulin receptor, insulin-like growth factor receptor, prostate specific membrane antigen, mesothelin, hepsin, an integrin, mucin (e.g., MUC16, etc.), a cell surface cluster of differentiation (CD) molecule, (e.g., CD20, CD22, CD30, CD33, CD44, CD56, etc.), proteins involved in immunological co-stimulation or co-inhibition or self-recognition such as CTLA4, PD-1, PD-L1, CD47, and any combination thereof.

In some embodiments of the methods of this invention, the target molecule can be a catalytic domain, regulatory domain and/or binding partner-interacting region of an intracellular, secreted, and/or membrane-bound protein [e.g., a growth factor, a cytokine, a secreted protein (e.g., VEGF, bFGF, EGF, IGF, PDGF, TGF, TNF, IgE and their respective receptors)], a kinase, a tyrosine kinase receptor (e.g., PI3K, AKT, MEK, EGFR, HER2, VEGFR, PDGFR, c-MET, insulin-like growth factor receptor, BRAF, etc.), a phosphatase (e.g., PTP1B, Cdc25, PTEN, SHP2), a protease (e.g., DPP-IV, caspase-3, cathepsin D, matriptase, a MT-MMP), an adhesion molecule (e.g., an integrin, EpCAM/TROP1, etc.), a protein target involved in an apoptotic pathway (e.g., Bc12, IAP, MDM2, HSP70, HSP90, etc.), a protein target involved in an epigenetic pathway (e.g., an acetyltransferase, a methyltransferase, a histone demethylase etc.), and a protein target involved in immunological co-stimulation or co-inhibition or self-recognition (e.g., CTLA4, PD-1, PD-L1, CD47), and any combination thereof.

In various embodiments, the targeting ligand of this invention has a target binding domain that binds the extracellular domain of the target molecule at or near a binding site of a native ligand of the target molecule such that binding of the targeting ligand modulates (e.g., disrupts, prevents, alters) the biological interaction between the native ligand and the target molecule (e.g., modulates the binding of EGF to the EGF receptor, etc.).

The present invention also provides a kit comprising a particle of this invention and/or a fusion protein of this invention and/or a particle coated with calmodulin on the surface and instructions for their use in the treatment of cancer in a subject and/or detection and/or localization of cancer cells and/or other diseased cells in a subject and/or diagnosis of cancer and/or other disorders (e.g., diabetes, asthma) in a subject.

Also provided herein is a composition comprising the particle of this invention, the nucleic acid molecule of this invention, the vector of this invention and/or the cell of this invention, as individual components or in any combination, in a pharmaceutically acceptable carrier.

### EXAMPLES

### Example 1

### A Universal, Reversible and Efficient Self-Assembly System to Confer Multiple Target-Binding Specificities to Nanoparticles

To make particles (e.g., nanoparticles or microparticles) useful for diagnosis, tumor imaging and targeted drug delivery, they need to be functionalized with specific targeting ligands. Herein is provided a universal, reversible and highly efficient self-assembly system that can confer particles with desired target-binding properties and multispecificity. This self-assembly system simplifies targeting conferment of any type of particles to a robust one-step chemical modification with calmodulin followed by a facile add-mix self-assembly of targeting ligands via a universal adapter peptide. In support of the proposed targeting method, binding specificity, reversibility and multispecificity of quantum dots modified using this system were carefully characterized.

Recent progress in sequencing entire cancer genomes has provided catalogues of all the mutations that are present in cancerous tissues. In the case of small cell lung cancer, for example, more than 23,000 mutations are exclusive to the diseased cells. Such a polygenetic nature clearly indicates that any effective cancer therapy should be based on targeting multiple pertinent signaling pathways, presumably by using a combination of targeting ligands (TLs) with different specificities. Nanoparticles are among some of the most promising platforms for targeted therapeutics. Surface-modified nanoparticles have been widely used in biomedical research, including for disease diagnosis, tumor imaging, cancer therapy and drug delivery. Restricting a cytotoxic anti-cancer drug to the diseased sites by TLs has the potential benefits of reducing serious side effects on healthy tissues, increasing local drug concentrations, and improving efficacy of treatment. Regardless of the materials used to compose the nanoparticles, a vital challenge is how to conjugate the TLs to the surface and how to precisely control the proportion of different ligands if multispecificity is desired. Due to the complicated nature of both nanomaterials and TLs, a facile and universal self assembly system that allows controllable loading of different targeting ligands to nanoparticles to acquire desired multispecificity would be highly desirable.

Ideally, the multispecificity and target-binding properties of nanoparticles would be easily self-assembled and tunable by simply changing the types and proportions of TLs added to it. Such nanoparticles loaded with multiple TLs could have greatly expanded applications to different tumor types and better therapeutic and diagnostic efficacies. However, conjugating nanoparticles with a single TL while still maintaining its target-binding ability is already challenging, let alone using multiple TLs, each possessing a unique specificity. In an attempt to simplify what otherwise would be a complex nanoparticle conjugation procedure, targeting ligands based on highly stable single protein domains were employed. Such single domain antibody mimics including Z domain-based affibody and FN3 domain-based monobody can be genetically engineered to introduce extra functional motifs and highly expressed in E. coli with very low cost.

The present invention describes the development of an efficient and universal self-assembly system that confers nanoparticles with the desired multispecificity for tumor diagnosis and therapeutics. The new method, summarized in FIG. 1, involves a universal acceptor (UA), calmodulin, and a universal adaptor peptide (UAP). This is a calmodulin-binding peptide that is present in Nature or isolated from a combinatory library as reported in Biochemistry, 2007, 46 (35), pp 10102-10112. To list just a few, nonlimiting examples for UAP from natural organisms include R R K W Q K T G H A V R A I G R L S SM (SEQ ID NO:12) from MLCK; NSAFVERVRKRGFEVV (SEQ ID NO:13) from HSP90; WSRIASLLHRKSAKQCKAR (SEQ ID NO:14) from CDC5-L; YEAHKRLGNRWAEIAKLLP (SEQ ID NO:15) from MYBL1; KEVIRNKIRAIGKMARVFSV (SEQ ID NO:16) from PPP3C, and ELRSLWRKAIHQQILLLR (SEQ ID NO:17) from TBC1. Nonlimiting examples of UAP from combinatorial libraries include KSIIQRNLRWNKFKRFYQD (SEQ ID NO:18); NILRQEVMKMGPAKDTVRN (SEQ ID NO:19); VKLRQRVTLAKRVAVNLNY (SEQ ID NO:44); LRLVPRIKALNKVQVKNHN (SEQ ID NO:26); WINNVRLRIHTKRWLLKSNH (SEQ ID NO:22); and WHKVFIRRQSKKLVYNTIKN (SEQ ID NO:23).

A 17-residue peptide fragment of human myosin light-chain kinase (MLCK) was used as one example. The nanoparticle is coated with the UA using NHS-ester chemistry. Recombinant expression is used to generate the TLs with the UAP fused to their carboxy termini. The resulting TL-UAP fusion proteins can be linked to the calmodulin-coated nanoparticles by mixing the two components in the presence of Ca²⁺, which is abundant in plasma and cell media. This method allows for efficient and stoichiometric self-assembly of one TL with one calmodulin. Since the binding of the TL to calmodulin occurs through the C-terminal UAP and all the TLs used are based on a single protein domain, there should be no bias in bonding affinity for any particular TL.

To demonstrate the universality of this UA-UAP system, three UAP-containing TLs (TL-UAPs) were engineered based on the reported Z-domain derived Z^{EGFR} and Z^{HER2} and FN3-domain derived FN3^{αvβ3} (see procedures in supplement). These three TLs specifically bind to EGFR, HER2 and integrin αvβ3, respectively, with high affinity. The three receptors are clinically validated cancer biomarkers that are overexpressed in a wide variety of cancers.

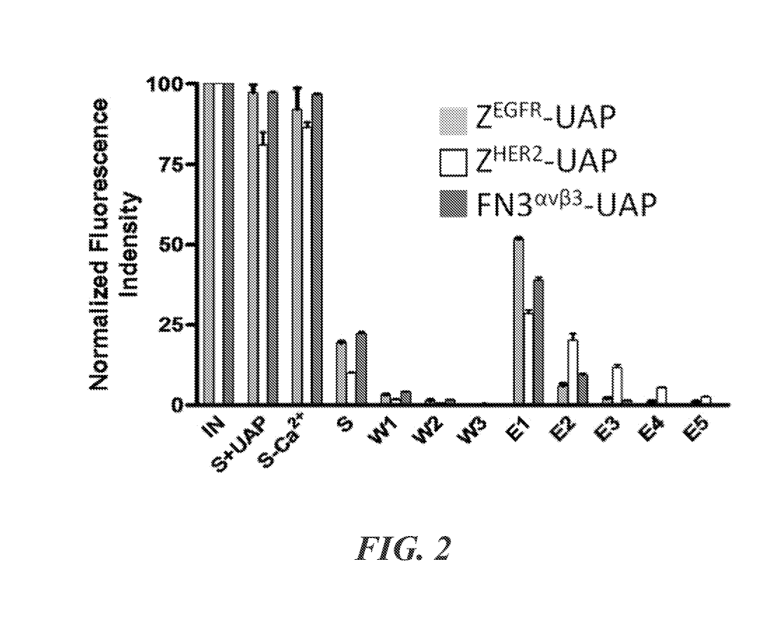

Studies were conducted to demonstrate the assembly efficiency, specificity and reversibility of the binding between the TL-UAPs and the UA. For this purpose, calmodulin-coated agarose beads and FITC-labeled TL-UAPs were used and fluorescence was measured as an indicator of the extent of binding between the TL-UAPs and UA (calmodulin). The results illustrated in FIG. 2 indicate that the TL-UAPs were not captured by the calmodulin-agarose beads when Ca²⁺ was not present (lane S−Ca²⁺) or when an excessive amount of the free MLCK peptide (the UAP used) was included as a competitor (lane S+UAP). In the presence of Ca²⁺, approximately 80% of the total TL-UAPs were captured by the beads (lane S). Such non-covalent self-immobilization is very strong and robust. Only a trace amount of the immobilized TL-UAPs was stripped by extensive and stringent washing (lanes W1 to W3), consistent with the low picomolar affinity between Ca²⁺/calmodulin and MLCK-derived UAP. The immobilization of such UAP-containing TLs is reversible, with about 90% of the captured TL-UAPs being released simply by including 1 mM EGTA in the same washing buffer (lanes E1 to E5). This highly specific binding and very mild elution process can be repeated several times without losing target-binding ability.

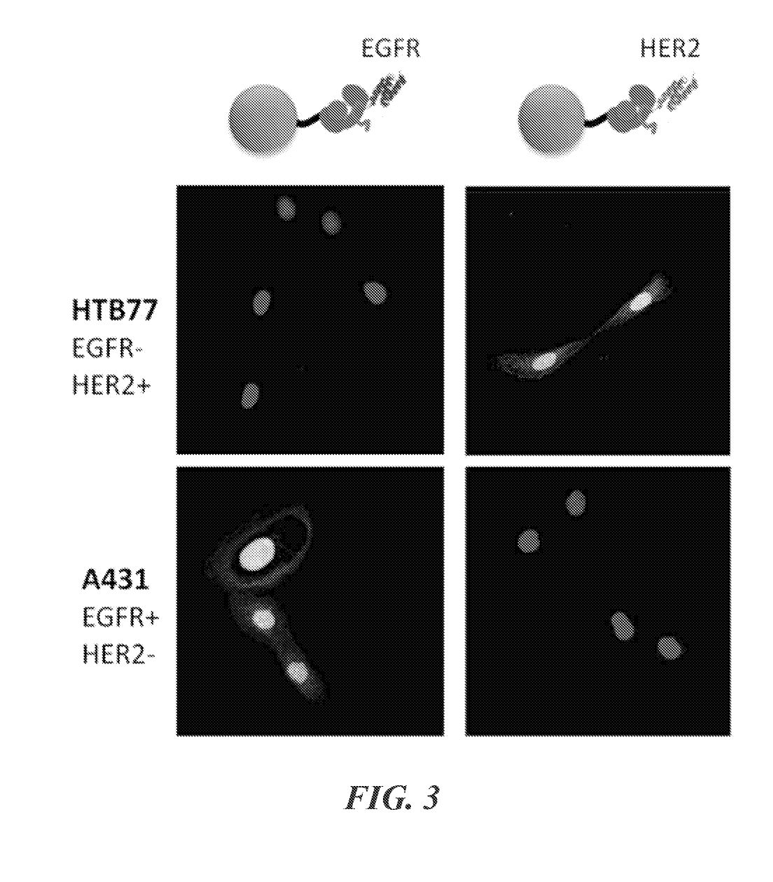

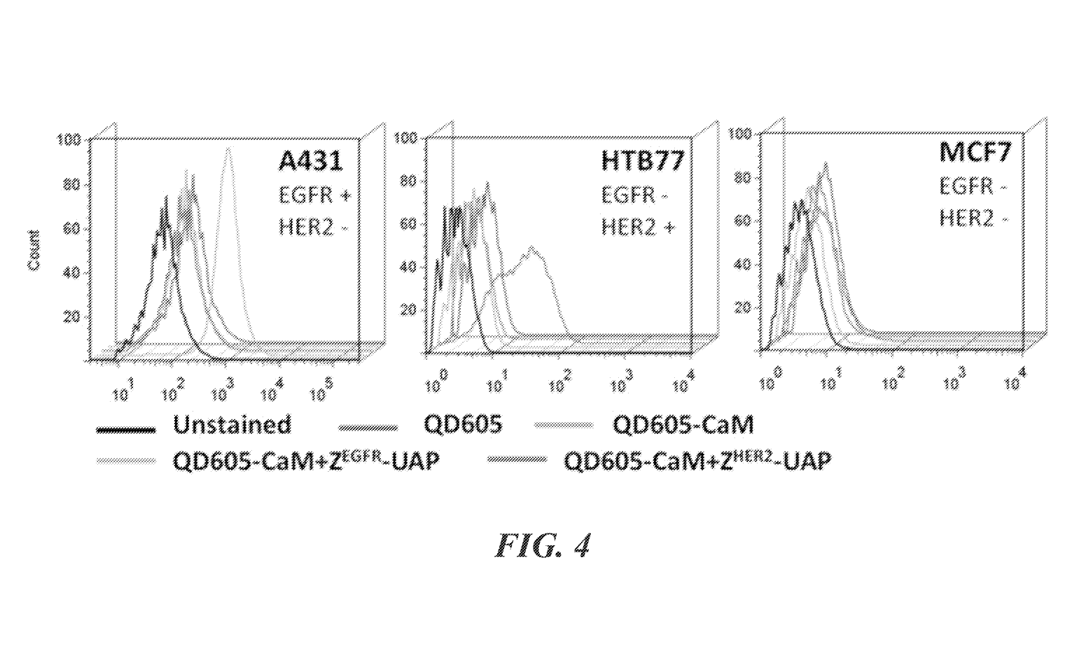

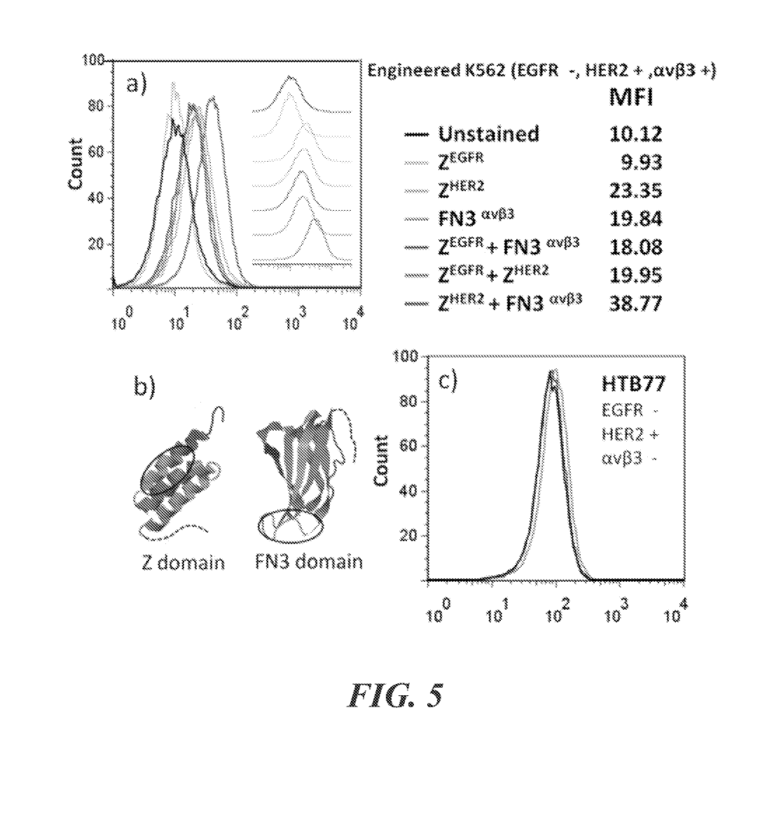

The same self-assembly of multiple targeting ligands was expanded to calmodulin-conjugated quantum dots (QD605-CaM) for further characterization and cellular binding analysis. Calmodulin-coated quantum dots were generated by a simple chemical conjugation through NHS chemistry and used for self-assembly with non-fluorescent Z^{EGFR}-UAP or Z^{HER2}-UAP separately. The resulting quantum dots with the desired TL(s) were applied to biomarker-positive or biomarker-negative cancer cells. As shown in FIG. 3, A431 cells (EGFR+, HER2−) were stained by QD605-CaM@Z^{EGFR}-UAP but not by QD605-CaM@Z^{HER2}-UAP, whereas HTB77 cells (EGFR−, HER2+) were only stained by QD605-CaM@Z^{HER2}-UAP. These results demonstrate that neither the fusion of the UAP to the TLs nor the tethering of the TL-UAPs to QD-bound CaM disrupts the binding specificity of the TLs. Such results were further confirmed by flow cytometry experiments, in which more controls and a negative MCF7 cell line (EGFR−, HER2−) were introduced (FIG. 4). Studies were then carried out to determine whether the UA-UAP system allows for readily conferring multiple target-binding specificities to nanoparticles by loading a mixture of two different TLs. To address this, QD605-CaM was mixed with a TL combination Z^{EGFR}-UAP+Z^{HER2}-UAP, Z^{EGFR}-UAP+FN3^{αvβ3}-UAP, or Z^{HER2}-UAP+FN3^{αvβ3}-UAP, respectively. The resulting TLs-conjugated nanoparticles, as well as the TLs alone, were individually incubated with an engineered leukemia cell line K562 (EGFR−, HER2+, αvβ3+). As shown in FIG. 5A, any combination containing a TL that recognizes either αvβ3 or HER2 gives a signal shift in flow cytometry distribution compared to the untreated cells. When the combination contains TLs that recognize αvβ3 and HER2, respectively, the signal was strongest. By analyzing the Median Fluorescence Intensity (MFI), it was found that the combination of two valid TLs with corresponding biomarkers on the cell surface (i.e., FN3^{αvβ3} and Z^{HER2}) resulted in a signal approximately 2 times higher than when only one TL was used, and 4 times higher than when no TL or an invalid TL (without corresponding biomarker on the cell surface) is used. These results indicate that QDs with bispecificity can be readily prepared by a facile one-step add-mix, providing an apparent additive effect of valid targeting ligands. Versatility of UAP-CaM system was also well demonstrated in this case. Due to the active sites of both Z domain and FN3 domain base TLs are closed to N terminus, the UAPs introduced at C terminus of respective protein rarely have chance to affect and be affected by TLs. Though the Z-domain is based on a 3-helix protein scaffold and FN3 on a totally different β-sandwich scaffold (FIG. 5B), both FN3^{αvβ3} and Z^{HER2} can be assembled on QDs through the C-terminal UAP without observable discrimination.

One concern is the possible exchange of the TLs on the nanoparticle surface, despite the unusually high affinity (<6 pM) between calmodulin and MLCK-derived UAP. To address this concern, QD605-CaM was saturated with Z^{EGFR}-UAP prior to incubation with Z^{HER2}-UAP or FN3^{αvβ3}-UAP for an additional one hour. If exchange occurs and free Z^{HER2}-UAP partially replaces pre-saturated Z^{EGFR}-UAP on the QDs, the expected observation is evidence of staining (i.e., a shift in the flow cytometry distribution) when the solution is applied to HER2-positive HTB77 cells on which HER2 is highly expressed. As shown in FIG. 5C, no such shift is observed, suggesting the exchange of TLs on the nanoparticle surface is under the detection limit.

Besides the universal, reversible and highly efficient self-assembly property, tunable multispecificity and high binding affinity, there are several additional advantages of this UA-UAP system. First, compared to the streptavidin-biotin and protein A/G-antibody systems which are widely used for the conjugation of TLs to nanoparticles, calmodulin is a highly stable monomeric protein completely conserved in all the mammals, with a molecular weight of only 16.7 kDa. Unlike the 53 kDa streptavidin from the bacteria Streptomyces avidinii and the 45 kDa protein A from the bacteria Staphylococcus aureus, calmodulin is ubiquitously expressed and very abundant in eukaryotes, constituting up to 0.1% of the total proteins in human cells, thus it rarely presents the problem of immunogenicity. Second, the tight binding of calmodulin to the TLs through the UAP is totally dependent on Ca²⁺. The concentration of Ca²⁺ in serum (mM scale) may stabilize TL conjugation which can be subsequently broken in the low Ca²⁺ environment of cytoplasm (nM scale) after internalization. Such Ca²⁺-concentration dependent, reversible self-assembly is compatible with future application of targeted drug delivery under physiological conditions by nanoparticles loaded with therapeutic agents. Third, Ca²⁺-saturated calmodulin can still retain its native conformation even in the presence of 2 M urea or at 90° C., with a high degree of reversibility of the unfolding process. This unusually high stability would facilitate chemical modification of many different nanoparticles, making the system feasible in even harsh conditions.

In conclusion, the present invention provides a universal, reversible and highly efficient self-assembly UA-UAP system, that allows multispecific targeting capabilities to be readily conferred to nanoparticles. The assembly efficiency, specificity and reversibility with specific TLs of different types are demonstrated herein. These studies have shown that complicated protein-nanoparticle conjugation chemistry can be replaced by a robust one-step chemical conjugation, followed by a facile add-mix self-assembly process that can be readily scaled up if needed. Mono-, di- or potential tri-specificity can be conferred to nanoparticles merely by tuning the composition and proportion of different TLs used. This method of targeting conferment can be used on many different nanoparticle and microparticle platforms, making it useful in areas as diverse as drug-delivery and cancer diagnosis.

Reagents.

Calmodulin agarose beads were purchased from Agilent. Calmodulin and calmodulin inhibitory peptide were purchased from CalBioChem. Quantum dots 605 ITK amino (PEG) were purchased from Invitrogen. Bis(Sulfosuccinimidyl) suberate (BS₃) was obtained from Pierce. Co²⁺-NTA Talon resin was obtained from Clontech. Restriction enzymes were obtained from NEB and other chemical reagents were from Sigma.

Targeting Ligand Expression and Purification.

Expression plasmids containing the targeting ligands were constructed by integrating the sequences that code for a target-binding domain, a flexible linker, and a calmodulin-binding motif (UAP), respectively, through a megaprimer PCR and cloning into the pET28b vector between Nco I/Xho I restriction sites. The final expression vectors were verified by sequencing. E. coli strain Rosetta (DE3) competent cells were transformed with the corresponding plasmids. One clone was picked up and grown at 37° C. overnight in 5 mL LB media supplemented with kanamycin (50 ug mL⁻¹) and chloramphenicol (34 ug mL⁻¹). 1 Liter of LB media was inoculated with 5 ml overnight cultures. The cells were grown until the OD₆₀₀ reached 0.8. The expression of recombinant proteins was induced by adding 1 mM IPTG at 37° C. for an additional 6 h of culturing and harvested by centrifugation (8000 g, 10 min, 4° C.).

To purify a recombinant protein of interest, the cell pellets were suspended in 20 mL binding buffer (50 mM sodium phosphate pH7.5, 300 mM NaCl, 10 mM imidazole, 5% glycerol, 5 mM β-mercaptoethanol, and 1 mM PMSF) and disrupted by sonication. After centrifugation at 16,000 g for 10 min at 4° C., the supernatant was loaded to a column containing Co²⁺-NTA Talon resin. After washing with the binding buffer, the bound protein was eluted with an elution buffer (50 mM sodium phosphate pH7.5, 300 mM NaCl, and 150 mM imidazole). After changing the buffer to PBS (pH 7.4), the purity and homogeneity of the purified proteins were estimated by SDS-PAGE, and the concentrations measured by the BCA method. The purified proteins are stored at −20° C. by supplementing glycerol to a final concentration of 25% (v/v) prior to final use.

Fluorescence Labeling of the Targeting Ligands.

The Z^{EGFR}-UAP, Z^{HER2}-UAP and FN3^{αvβ3}-UAP were labeled at exposed lysine residues with FITC. In general, 100 μg of a relevant protein in 100 μl PBS (pH 7.4) buffer was reacted with a 10 molar excess of FITC at room temperature in dark with gentle shaking for 2 h. The reaction was quenched by adding 5 μL of 1 M glycine (pH 9.0) followed by additional 15 min incubation. Excess FITC and glycine were removed by passing through a NAP-5 column pre-equilibrated with PBS (pH 7.4). Extensive dialysis was performed to remove any unreacted residual fluorophore. Conjugation of calmodulin to nanoparticles. 133.6 μg calmodulin was first dissolved in 50 μL PBS (pH 7.4). 25 μL of 8 μM Quantum dots 605 ITK amino (PEG) was activated by incubating with 5 μL 10 mM BS₃ at room temperature for 30 min followed by removing unreacted BS₃ using NAP-5 column. The colored fraction was mixed with previously prepared calmodulin solution and the mixture was gently rotated for 2 h at room temperature. The reaction was quenched by adding glycine solution to a final concentration of 50 mM followed by 15 min incubation. The QD605-CaM conjugates were purified by using 100 kD Vivaspin ultrafiltration tube (GE Healthcare) and the buffer was changed to 50 mM borate (pH 8.3) according to manufacturer's instructions. This typically takes 5 or 6 rounds. QD605-CaM conjugates were stored at 4° C. protected from light. Concentration of QD605-CaM was estimated with a fluorometer using original QD605 solution as a standard.

Self-Assembly of the Targeting Ligand(s) on Nanoparticles.

To load targeting ligand(s) of interest to nanoparticles, a solution of targeting ligand (one or a mixture of more than one at desired ratios) and a solution of QD605-CaM were prepared in PBS (pH 7.4) in the presence of 1 mM CaCl₂ just before use. The self-assembly of the targeting ligand(s) on nanoparticle surface was performed by mixing above two components at a 1:10 molar ratio for 10 min at room temperature.

Cell Culture.

The EGFR-expressing squamous carcinoma cell line A431 was cultured at 37° C. in a humidified 5% CO₂ environment in Dulbecco's-modified Eagle's medium supplemented with 10% fetal bovine serum (FBS) and 2 mM L-glutamine. The HER2-expressing human ovary adenocarcinoma cell line HTB77 was cultured in McCoy's 5a medium supplemented with 10% FBS and 2 mM L-glutamine. Estrogen-dependent mammary adenocarcinoma cell line MCF7 (low expression level of EGFR and HER2) was maintained in Eagle's Minimum Essential Medium supplemented 10% FBS, 2 mM L-glutamine and 0.01 mg/mL human recombinant insulin (GIBCO). Engineered human leukemia cells K562/αvβ3 (αvβ3+), were obtained from Upstate Medical University. This cell line was maintained in Iscove's Modified Dulbecco's Medium (IMDM) supplemented with 10% FBS, 2 mM L-glutamine, 300 μg/ml G418 (Invitrogen) and cultured as described above.

In Vitro Targeting Ligand Capture Assay.

100 μg of a FITC-labeled targeting ligand (Z^{EGFR}-UAP, Z^{HER2}-UAP, FN3^{αvβ3}-UAP) was incubated with 100 μL of CaM-Agarose beads in 500 μL of binding buffer (1×PBS pH7.4 supplemented with 10 mM β-mercaptoethanol, 1 mM magnesium acetate, 1 mM imidazole, and 1 mM CaCl₂) for 1h at 4° C. The controlled experiments were conducted in the presence of 15 mM free MLCK peptide as competitor or in the absence of 1 mM CaCl₂. Incubated slurry was loaded into a 0.8 mL Handee centrifuge column (Pierce). After collecting flowthrough at 1500 rpm/min for 1 min, the resins were washed with 500 μL of binding buffer three times. Captured targeting ligands were eluted with 500 μl of elution buffer (1×PBS pH7.4 supplemented with 10 mM β-mercaptoethanol and 4 mM EGTA) for five times. Fluorescence of each fraction was measured by plate reader (Biotek FLx800 Ex 488 nm, Em 525 nm). All experiments were performed in triplicates.

FACS analysis. Subconfluent cells were dissociated from flask with the non-enzyme dissociation buffer (Sigma), followed by washing twice with FACS binding buffer (Hank's Buffered Salt Solution supplemented with 2% BSA and 1 mM CaCl₂). Each analysis was performed in triplicates by using 3×10⁵ cells each time. Briefly, QD605-CaM was mixed with targeting ligand(s) to a final concentration of 10 nM and 100 nM, respectively, in 100 μL FACS binding buffer. The mixture was preincubated at room temperature for 30 min before the addition of 100 μL of cell suspension. After further incubation with gentle shaking at 4° C. for 30 min, cells were washed twice with ice-cold FACS binding buffer and analyzed by BD FACS Canto flow cytometer (BD Biosciences).

Confocal Analysis.

Cells were cultured on poly-lysine treated cover slides overnight. After a brief washing with PBS, cells were fixed with 3.7% formaldehyde at room temperature for 10 min. The slides were then sequentially washed with PBS three times (10 min each), blocked with PBS (with 2% BSA) for 1 hour, and washed again with PBS three times, followed by incubation with 5 nM QD605-CaM with targeting ligand(s) assembled in PBS (2% BSA, 1 mM CaCl₂) at room temperature for 30 min. After staining, slides were extensively washed with PBS four times (15 min each). Air dried slides were mounted in a DAPI containing anti-fade solution (Vector Lab). Images were acquired by using Zeiss LSM710 Spectral Confocal Laser Scanning Microscopy at UNC Lineberger Cancer Center.

All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the list of the foregoing embodiments and the appended claims.

1. A particle, which can be a nanoparticle or a microparticle, comprising calmodulin attached to an exterior surface, wherein the calmodulin is attached to a fusion protein comprising a targeting ligand and a carboxy-terminal or amino-terminal calmodulin binding peptide.

2. The particle of claim 1, wherein the calmodulin is attached to the exterior surface using hydrophobic noncovalent interaction or covalent linkage based on amine/carboxylate chemistry, thiol/maleimide chemistry, and/or disulfide chemistry.

3. The particle of claim 1, wherein the calmodulin tightly binds to a calmodulin-binding peptide fused at the amino- or carboxy-terminal of a targeting ligand in the presence of calcium (Ca²⁺).

4. The particle of claim 1, wherein the calmodulin-binding peptide at the amino- or carboxy-terminal of a targeting ligand is selected from the group consisting of a peptide comprising the amino acid sequence RRKWQKTGHAVRAIGRLSSM (SEQ ID NO:12) from smooth muscle myosin light-chain kinase; a peptide comprising the amino acid sequence KRRWKKNFIAVSAANRFKKI (SEQ ID NO:24) from skeletal muscle MLCK; a peptide comprising the amino acid sequence RRKLKGAILTTMLATR (SEQ ID NO:25) from Ca²⁺/CaM-dependent protein kinases, a peptide comprising the amino acid sequence IPSWTTVILVKSMLRKRSFGNPF (SEQ ID NO:26) from Ca²⁺/CaM-dependent protein kinase, a peptide comprising the amino acid sequence QILWFRGLNRIQTQIRVVNA (SEQ ID NO:27) from plasma membrane calcium-ATPase, a peptide comprising the amino acid sequence ITRIQAQSRGVLARMEYKKL (SEQ ID NO:28) from beta myosin heavy chain, a peptide comprising the amino acid sequence ATLIQKIYRGWRCRTHYQLM (SEQ ID NO:29) from myosin IA, a peptide comprising the amino acid sequence AAKIQASFRGHMARKKIKSG (SEQ ID NO:30) from neurogranin, a peptide comprising the amino acid sequence AIIIQRAYRRYLLKQKVKKV (SEQ ID NO:31) from voltage-gated sodium channel, a peptide comprising the amino acid sequence LGLVQSLNRQRQKQLLNENN (SEQ ID NO:32) or RLLWQT AVRHITEQRFIHGHR (SEQ ID NO:33) from adenylyl cyclase, a peptide comprising the amino acid sequence NEELRAIIKKIWKRTSMKLL (SEQ ID NO:34) from L-type Ca²⁺ channel, a peptide comprising the amino acid sequence MRSVLISLKQAPLVH (SEQ ID NO:35) from clathrin light chain A, a peptide comprising the amino acid sequence ARKEVIRNKIRAIGKMARVFSVLR (SEQ ID NO:36) from calcineurin A, a peptide comprising the amino acid sequence KPKFRSIVHAVQAGIFVERMFRR (SEQ ID NO:37) from phosphodiesterase 1B, a peptide comprising the amino acid sequence SYEFKSTVDKLIKKTNLALV (SEQ ID NO:38) from sodium/calcium exchanger (SLC8A), a peptide comprising the amino acid sequence HTLIKKDLNMVVSAARISCG (SEQ ID NO:39) from titin, a peptide comprising the amino acid sequence EIRFTVLVKAVFFASVLMRK (SEQ ID NO:40) from inducible nitric oxide synthase, a peptide comprising the amino acid sequence AIGFKKLAEAVKFSAKLMGQ (SEQ ID NO:41) from neuronal nitric oxide synthase, a peptide comprising the amino acid sequence ASPWKSARLMVHTVATFNSIK (SEQ ID NO:42) from spectrin, a peptide comprising the amino acid sequence NSAFVERVRKRGFEVV (SEQ ID NO:13) from heat shock protein 90; a peptide comprising the amino acid sequence WSRIASLLHRKSAKQCKAR (SEQ ID NO:14) from cell cycle serine/threonine-protein kinase 5-like; a peptide comprising the amino acid sequence YEAHKRLGNRWAEIAKLLP (SEQ ID NO:15) from V-Myb Myeloblastosis Viral Oncogene Homolog like-1; a peptide comprising the amino acid sequence QKEVLITWDKKLLNCRAKIR (SEQ ID NO:43) from TBC1; a peptide comprising the amino acid sequence KSIIQRNLRWNKFKRFYQD (SEQ ID NO:18); a peptide comprising the amino acid sequence NILRQEVMKMGPAKDTVRN (SEQ ID NO:19); a peptide comprising the amino acid sequence VKLRQRVTLAKRVAVNLNY (SEQ ID NO:44); a peptide comprising the amino acid sequence LRLVPRIKALNKVQVKNHN (SEQ ID NO:21); a peptide comprising the amino acid sequence WINNVRLRIHTKRWLLKSNH (SEQ ID NO:22); a peptide comprising the amino acid sequence WHKVFIRRQSKKLVYNTIKN (SEQ ID NO:23) and any combination thereof.

5. The particle of claim 1, wherein the targeting ligand is a single chain polypeptide of a V_{H} or V_{L} domain of an antibody, a peptide or protein derived from a binding and/or framework region of an antibody, a single domain antibody mimic based on non-immunoglobulin scaffolds (such as FN domain-based monobody, Z domain-based affibody, DARPIN), a short target-binding peptide containing natural and/or unnatural amino acids, which specifically binds to the extracellular domain of a cell surface receptor, and any combination thereof.

6. The particle of claim 5, wherein the targeting ligand is selected from the group consisting of FN3^{VEGFR}, FN3^{αvβ3}, FN3^{EGFR}, FN3^{HER2}, FN3^{HER3}, FN3^{PSMA}, FN3^{GRP78}, FN3^{c-MET}, FN3^{IGFIR}, Z^{EGFR}, Z^{HER2}, Z^{HER3}, and any combination thereof.

7. The particle of claim 1, wherein the particle comprises a therapeutic agent.

8. The particle of claim 1, wherein the particle comprises a detectable agent.

9. The particle of claim 1, comprising two different fusion proteins wherein each different fusion protein comprises a different targeting ligand and a carboxy-terminal or amino terminal calmodulin binding peptide.

10. The particle of claim 1, comprising three different fusion proteins wherein each different fusion protein comprises a different targeting ligand and a carboxy-terminal or amino-terminal calmodulin binding peptide.

11. A method of making a particle comprising a calmodulin/fusion protein complex by a self-assembly process in the presence of calcium.

12. A method of delivering a therapeutic agent to a cell of a subject, comprising administering to the subject the particle of claim 1, wherein the particle comprises a therapeutic agent and where the particle further comprises a targeting ligand specific for the cell of the subject to which the therapeutic agent is to be delivered.

13. A method of detecting the presence and/or location of a target cell in a subject, comprising administering to the subject the particle of claim 1, wherein the particle comprises a detectable agent and where the particle further comprises a targeting ligand specific for the target cell.

14. A method of treating cancer in a subject in need thereof, comprising administering to the subject the particle of claim 1, wherein the particle comprises a chemotherapeutic agent and/or anti-cancer agent and wherein the particle further comprises a targeting ligand specific for a cancer cell of the subject to which the chemotherapeutic agent and/or anti-cancer agent is to be delivered, thereby treating cancer in the subject.

15. A method of diagnosing cancer in a subject, comprising administering to the subject the particle of claim 1, wherein the targeting ligand on the particle is specific for a target molecule on a cancer cell in the subject and the particle further comprises an imaging molecule and/or detectable molecule, whereby the targeting ligand binds the target molecule on a cancer cell in the subject and the imaging molecule is visualized and/or the detectable molecule is detected on a cancer cell in the subject, thereby diagnosing cancer in the subject.


SEQUENCE LISTING (44 sequences)

SEQ ID NO: 1 - protein, 11 aa, Artificial
  Linker sequence
GPQPQPKPQPK

SEQ ID NO: 2 - protein, 5 aa, Artificial
  Linker sequence
GGGGS

SEQ ID NO: 3 - protein, 15 aa, Artificial
  Linker sequence
TPPTPSPSTPPTPSP

SEQ ID NO: 4 - protein, 16 aa, Artificial
  Linker sequence
EFPKPSTPPGSSGGAP

SEQ ID NO: 5 - protein, 16 aa, Artificial
  Linker sequence
PQPQPQPKPQPKPEPE

SEQ ID NO: 6 - protein, 4 aa, Artificial
  Linker sequence
GGGS

SEQ ID NO: 7 - protein, 6 aa, Artificial
  Linker sequence
GSGSGS

SEQ ID NO: 8 - protein, 7 aa, Artificial
  Linker sequence
TPPTPSP

SEQ ID NO: 9 - protein, 6 aa, Artificial
  Linker sequence
PQPQPK

SEQ ID NO: 10 - protein, 6 aa, Artificial
  Linker sequence
PQPQPE

SEQ ID NO: 11 - protein, 10 aa, Artificial
  Linker sequence
PEPEPQPQGG

SEQ ID NO: 12 - protein, 20 aa, Artificial
  Calmodulin binding peptide sequence
RRKWQKTGHAVRAIGRLSSM

SEQ ID NO: 13 - protein, 16 aa, Artificial
  Calmodulin binding peptide sequence
NSAFVERVRKRGFEVV

SEQ ID NO: 14 - protein, 19 aa, Artificial
  Calmodulin binding peptide sequence
WSRIASLLHRKSAKQCKAR

SEQ ID NO: 15 - protein, 19 aa, Artificial
  Calmodulin binding peptide sequence
YEAHKRLGNRWAEIAKLLP

SEQ ID NO: 16 - protein, 20 aa, Artificial
  Calmodulin binding peptide sequence
KEVIRNKIRAIGKMARVFSV

SEQ ID NO: 17 - protein, 18 aa, Artificial
  Calmodulin binding peptide sequence
ELRSLWRKAIHQQILLLR

SEQ ID NO: 18 - protein, 19 aa, Artificial
  Calmodulin binding peptide sequence
KSIIQRNLRWNKFKRFYQD

SEQ ID NO: 19 - protein, 19 aa, Artificial
  Calmodulin binding peptide sequence
NILRQEVMKMGPAKDTVRN

SEQ ID NO: 20 - protein, 20 aa, Artificial
  Calmodulin binding peptide sequence
WVKLRQRVTLAKRVAVNLNY

SEQ ID NO: 21 - protein, 19 aa, Artificial
  Calmodulin binding peptide sequence
LRLVPRIKALNKVQVKNHN

SEQ ID NO: 22 - protein, 20 aa, Artificial
  Calmodulin binding peptide sequence
WINNVRLRIHTKRWLLKSNH

SEQ ID NO: 23 - protein, 20 aa, Artificial
  Calmodulin binding peptide sequence
WHKVFIRRQSKKLVYNTIKN

SEQ ID NO: 24 - protein, 20 aa, Artificial
  Calmodulin binding peptide sequence
KRRWKKNFIAVSAANRFKKI

SEQ ID NO: 25 - protein, 16 aa, Artificial
  Calmodulin binding peptide sequence
RRKLKGAILTTMLATR

SEQ ID NO: 26 - protein, 23 aa, Artificial
  Calmodulin binding peptide sequence
IPSWTTVILVKSMLRKRSFGNPF

SEQ ID NO: 27 - protein, 20 aa, Artificial
  Calmodulin binding peptide sequence
QILWFRGLNRIQTQIRVVNA

SEQ ID NO: 28 - protein, 20 aa, Artificial
  Calmodulin binding peptide sequence
ITRIQAQSRGVLARMEYKKL

SEQ ID NO: 29 - protein, 20 aa, Artificial
  Calmodulin binding peptide sequence
ATLIQKIYRGWRCRTHYQLM

SEQ ID NO: 30 - protein, 20 aa, Artificial
  Calmodulin binding peptide sequence
AAKIQASFRGHMARKKIKSG

SEQ ID NO: 31 - protein, 20 aa, Artificial
  Calmodulin binding peptide sequence
AIIIQRAYRRYLLKQKVKKV

SEQ ID NO: 32 - protein, 20 aa, Artificial
  Calmodulin binding peptide sequence
LGLVQSLNRQRQKQLLNENN

SEQ ID NO: 33 - protein, 21 aa, Artificial
  Calmodulin binding peptide sequence
RLLWQTAVRHITEQRFIHGHR

SEQ ID NO: 34 - protein, 20 aa, Artificial
  Calmodulin binding peptide sequence
NEELRAIIKKIWKRTSMKLL

SEQ ID NO: 35 - protein, 15 aa, Artificial
  Calmodulin binding peptide sequence
MRSVLISLKQAPLVH

SEQ ID NO: 36 - protein, 24 aa, Artificial
  Calmodulin binding peptide sequence
ARKEVIRNKIRAIGKMARVFSVLR

SEQ ID NO: 37 - protein, 23 aa, Artificial
  Calmodulin binding peptide sequence
KPKFRSIVHAVQAGIFVERMFRR

SEQ ID NO: 38 - protein, 20 aa, Artificial
  Calmodulin binding peptide sequence
SYEFKSTVDKLIKKTNLALV

SEQ ID NO: 39 - protein, 20 aa, Artificial
  Calmodulin binding peptide sequence
HTLIKKDLNMVVSAARISCG

SEQ ID NO: 40 - protein, 20 aa, Artificial
  Calmodulin binding peptide sequence
EIRFTVLVKAVFFASVLMRK

SEQ ID NO: 41 - protein, 20 aa, Artificial
  Calmodulin binding peptide sequence
AIGFKKLAEAVKFSAKLMGQ

SEQ ID NO: 42 - protein, 21 aa, Artificial
  Calmodulin binding peptide sequence
ASPWKSARLMVHTVATFNSIK

SEQ ID NO: 43 - protein, 20 aa, Artificial
  Calmodulin binding peptide sequence
QKEVLITWDKKLLNCRAKIR

SEQ ID NO: 44 - protein, 19 aa, Artificial
  Calmodulin binding peptide sequence
VKLRQRVTLAKRVAVNLNY